(19) United States
(12) Patent Application Publication
(10) Pub. No.: US 2026/0240654 A1
(43) Pub. Date: Aug. 20, 2026

(54) HEART VALVE REPAIR

(71) Applicant: Edwards Lifesciences Corporation, Irvine, CA (US)

(72) Inventors: Nima V. Nia, Mission Viejo, CA (US); Hengchu Cao, Irvine, CA (US); Bingquan Su, Irvine, CA (US); Douglas Thomas Dominick, Irvine, CA (US); Sakyasingh Tripathy, San Diego, CA (US)

(21) Appl. No.: 19/642,544

(22) Filed: Apr. 8, 2026

Related U.S. Application Data
(63) Continuation of application No. 17/507,709, filed on Oct. 21, 2021, now Pat. No. 12,622,784.
Application No. 17/507,709 is a continuation of application No. PCT/US2020/027789, filed on Apr. 10, 2020.
(60) Provisional application No. 62/836,839, filed on Apr. 22, 2019.

(51) Int. Cl. A61F 2/24

(52) U.S. Cl. CPC A61F 2/2463; A61F 2/2454; A61F 2/246; A61F 2220/0016; A61F 2220/0075

(57) ABSTRACT

In a method of treating a heart valve, a catheter is inserted into a heart. A device is deployed from the catheter into the heart. The device has a plurality of paddles that each include an inner paddle portion and an outer paddle portion. The paddles are moved to a partially open position. The device is moved such that the inner paddle portions engage leaflets of a native heart valve. The leaflets are pierced and one or more anchors are moved through the leaflets. The device is removed, leaving the one or more sutures attached to the leaflets.

### RELATED APPLICATION

This application is a continuation of US Patent Application serial no. 17/507,709, filed on October 21, 2021, which is a continuation of PCT application no. PCT/US2020/027789, filed on Apr 10, 2020, which claims priority to U.S. Provisional Application No. 62/836,839, filed on April 22, 2019, entitled HEART VALVE REPAIR SYSTEM, the disclosures of which are all hereby incorporated by reference in their entireties.

### BACKGROUND

### FIELD

The present disclosure generally relates to the field of medical devices and procedures.

### DESCRIPTION OF RELATED ART

The native heart valves (i.e., the aortic, pulmonary, tricuspid, and mitral valves) of the human heart aid in the circulation blood through the cardiovascular system. These heart valves can be damaged, and thus rendered less effective, by congenital malformations, inflammatory processes, infectious conditions, or disease.

### SUMMARY

The disclosure describes, in one aspect, an implantable valve repair system for repairing a native valve of a patient. The implantable valve repair system for repairing a native valve of a patient includes an implantable prosthetic device and a connector. The implantable prosthetic device is configured to attach to two leaflets of the native valve of the patient. The connector is separate from the implantable prosthetic device and is also configured to attach to the two leaflets of the native valve of the patient. The implantable prosthetic device is configured both to allow the implantable prosthetic device to remain attached to the native valve after the connector is attached to the native valve and to allow the implantable prosthetic device to be removed from the native valve after the connector is attached to the native valve.

In some implementations, the present disclosure relates to a heart valve repair system comprising an implantable prosthetic device configured to attach to at least two leaflets of a native heart valve of a patient and hold the at least two leaflets in a relatively fixed position, and a connector that is separate from the implantable prosthetic device, the connector including first and second tissue anchors and being configured to be attached to the at least two leaflets of the native valve of the patient when the at least two leaflets are held by the implantable prosthetic device in the relatively fixed position, the implantable prosthetic device being configured to be detached from the at least two leaflets and removed from the patient with the connector remaining attached to the at least two leaflets.

The implantable prosthetic device may include a plurality of paddles that each comprise an inner paddle portion and an outer paddle portion. In some embodiments, the implantable prosthetic device includes a coaption element. For example, the paddles can be connected to a distal end of the coaption element. The prosthetic device and the connector can be configured to close a gap in the native valve of the patient when the valve repair system is attached to the native valve. The connector can be configured to keep the gap in the native valve of the patient closed after the prosthetic device is removed.

The heart valve repair system can further comprise one or more additional connectors. The connector can comprise a first anchor configured to be positioned on a ventricular side of a first leaflet of the at least two leaflets, a second anchor configured to be positioned on a ventricular side of a second leaflet of the at least two leaflets, and a connecting portion including one or more suture lines coupled to the first and second anchors and configured to extend through the first leaflet and the second leaflet.

In some embodiments, the connecting portion comprises a first suture line extending from the first anchor through the first leaflet, a second suture line extending from the second anchor through the second leaflet, and a suture-locking device engaging both the first suture line and the second suture line to hold the first leaflet and the second leaflet together. For example, the suture-locking device can include a wedge and a locking nut. In some embodiments, nut is configured to threadably engage the wedge to capture the first suture line and the second suture line within the wedge. The wedge can include a first engagement surface configured to engage a first leaflet of the at least two leaflets and a second engagement surface configured to engage a second leaflet of the at least two leaflets.

In some embodiments, the connector comprises a shape-memory alloy. In some embodiments, the connector is one of a pledgeted suture or a staple. In some embodiments, the implantable prosthetic device includes a first anchor configured to embed into a first valve leaflet and a second anchor configured to embed into a second valve leaflet.

In some implementations, the present disclosure relates to a valve repair system comprising an implantable prosthetic device comprising a first and second paddles that are movable between an open position and a closed position, and a first and second tissue-gripping elements associated with the first and second paddles, respectively, each of the first and second tissue-gripping elements being configured to attach to a respective leaflet of a native heart valve of a patient. The valve repair system further comprises a connector configured to be implanted in first and second leaflets of the native heart valve of the patient when the implantable prosthetic device is gripped to the first and second leaflets and to allow for the implantable prosthetic device to be removed when the connector is implanted in the first and second leaflets, the connector comprising a first anchor configured to engage a first valve leaflet of the native heart valve of the patient, a second anchor configured to engage a second valve leaflet of the native heart valve of the patient, and a connecting portion attached to and extending between the first anchor and the second anchor.

Each of the first and second paddles can comprise an inner paddle portion and an outer paddle portion. In some embodiments, the implantable prosthetic device includes a coaption element. The connector can be configured to keep a gap in the native valve of the patient closed after the prosthetic device is removed. The valve repair system can further comprise two or more additional connectors. The connector can include one or more suture lines.

The connector may include a wedge and a locking nut, the locking nut configured to be attached to the wedge to capture a first suture and a second suture within the wedge. For example, the wedge can include a first engagement surface configured to engage the first valve leaflet and a second engagement surface configured to engage the second valve leaflet. The connector can be one of a pledgeted suture or a staple. In some embodiments, the first anchor is configured to embed into the first valve leaflet and the second anchor is configured to embed into the second valve leaflet.

In some implementations, the present disclosure relates to a method of repairing a heart valve. The method comprises implanting a leaflet-securing implant on first and second leaflets of a native heart valve of a patient, the leaflet-securing implant including two or more leaflet-engagement features configured to hold respective portions of the first and second leaflets in a relatively fixed position. The method further comprises, while the leaflet-securing implant is engaged with the first and second leaflets, implanting an edge-to-edge connector device in the native heart valve of the patient at least in part by engaging a first tissue anchor of the edge-to-edge connector device with the first leaflet and engaging a second tissue anchor of the edge-to-edge connector device with the second leaflet, the second tissue anchor being coupled to the first tissue anchor via one or more connecting sutures. The method further comprises, after implanting the edge-to-edge connector device in the native heart valve, decoupling the leaflet-securing implant from the first and second leaflets, withdrawing the leaflet-securing implant from the heart of the patient, and maintaining the edge-to-edge connector device implanted in the native heart valve.

The leaflet-securing implant can include a spacer configured to occupy at least a portion of a space between respective edges of the first and second leaflets of the native heart valve when the leaflet-securing implant is engaged with the first and second leaflets. In some embodiments, the method further comprises, while the leaflet-securing implant is engaged with the first and second leaflets, implanting one or more additional edge-to-edge connector devices in the native heart valve of the patient. In some embodiments, the first tissue anchor is a first end of a staple device, the second tissue anchor is a second end of the staple device opposite the first end, and the one or more connecting sutures are an intermediate portion of the staple device between the first end and the second end.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures can be drawn to scale for some embodiments, the figures are not necessarily drawn to scale for all embodiments. Embodiments and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a diastolic phase, in which the chordae tendineae are shown attaching the leaflets of the mitral and tricuspid valves to ventricle walls;

FIG. 4 illustrates a healthy mitral valve with the leaflets closed as viewed from an atrial side of the mitral valve;

FIG. 5 illustrates a dysfunctional mitral valve with a visible gap between the leaflets as viewed from an atrial side of the mitral valve;

FIG. 6 illustrates a tricuspid valve viewed from an atrial side of the tricuspid valve;

FIGS. 7-12 show an exemplary embodiment of an implantable prosthetic device being delivered and implanted within the native mitral valve;

FIGS. 13-18 shows the implantable prosthetic device of FIGS. 7-12 applying a suture and removing the device.

FIG. 19 shows a mitral valve with the leaflets sutured together in multiple locations as viewed from an atrial side of the mitral valve;

FIG. 20 illustrates a mitral valve with the leaflets attached together by an exemplary embodiment of an attachment device deployed by the prosthetic device;

FIG. 21 illustrates a mitral valve with the leaflets attached together by another exemplary embodiment of an attachment device deployed by the prosthetic device;

FIG. 22 illustrates a mitral valve with the leaflets attached together by another exemplary embodiment of an attachment device deployed by the prosthetic device; and

FIGS. 23-26 illustrates a mitral valve with an exemplary embodiment of a suture attaching the valve leaflets together.

### DETAILED DESCRIPTION

The following description refers to the accompanying drawings, which illustrate specific embodiments of the present disclosure. Other embodiments having different structures and operation do not depart from the scope of the present disclosure.

Exemplary embodiments of the present disclosure are directed to systems and methods for repairing a defective heart valve. It should be noted that various embodiments of native valve reparation devices and systems for delivery thereof are disclosed herein, and any combination of these options/features can be made unless specifically excluded. In other words, individual components of the disclosed devices and systems can be combined unless mutually exclusive or otherwise physically impossible.

As described herein, when one or more components are described as being connected, joined, affixed, coupled, attached, or otherwise interconnected, such interconnection may be direct as between the components or may be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, by congenital malformations, inflammatory processes, infectious conditions, or disease. Such damage to the valves can result in serious cardiovascular compromise or death. For many years, the definitive treatment for such damaged valves was surgical repair or replacement of the valve during open heart surgery. However, open-heart surgeries are highly invasive and are prone to many complications. Therefore, elderly and frail patients with defective heart valves may not be suitable candidates for open-heart operations. As an alternative, transvascular cardiac operations can be implemented for introducing and implanting prosthetic devices in a manner that is generally much less invasive than open heart surgery. One particular transvascular technique that can be used for accessing the native mitral and aortic valves is the trans-septal technique. The transseptal technique can involve inserting a catheter into the right femoral vein, up the inferior vena cava and into the right atrium. The septum is then punctured and the catheter passed into the left atrium. Access to the right atrium can be made via other blood vessels or access points. Furthermore, access to the aortic, tricuspid, mitral, or pulmonary valves in connection with any of the embodiments of the present disclosure can be made using a trans-aortic or trans-apical approach as well.

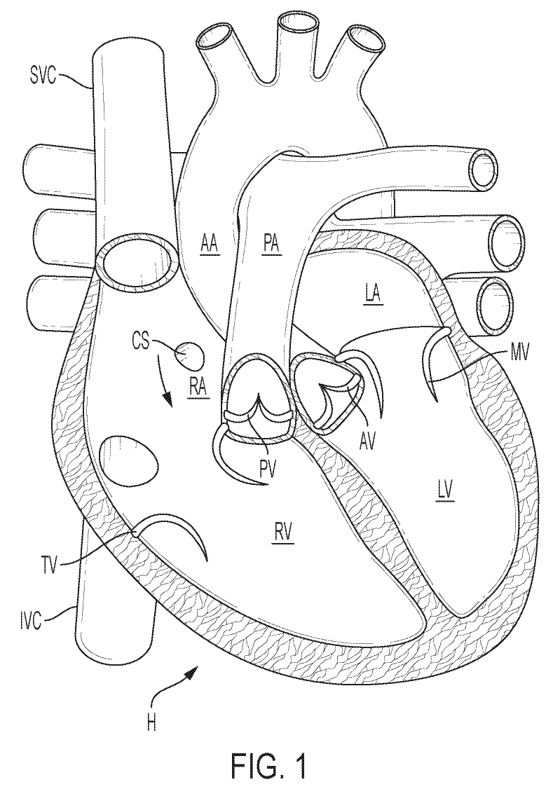

As described in greater detail below in connection with the description of FIG. 1, a healthy human heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve generally has a different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus can generally form a “D”-shaped, oval, or otherwise out-of-round cross-sectional shape having major and minor axes. The anterior leaflet can be larger than the posterior leaflet in some patients, forming a generally “C”-shaped coaptation boundary between the abutting edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the edges of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward/into the left atrium, a plurality of fibrous cords called chordae tendineae tether the leaflets to papillary muscles in the left ventricle.

Mitral regurgitation occurs when the native mitral valve fails to close properly and blood flows into the left atrium from the left ventricle during the systolic phase of heart contraction. Mitral regurgitation is a common form of valvular heart disease. Mitral regurgitation can have different causes, including leaflet prolapse, dysfunctional papillary muscles, or stretching or deformation of the mitral valve annulus resulting from, for example, dilation of the left ventricle. Mitral regurgitation at a central portion of the leaflets can be referred to as central jet mitral regurgitation, whereas mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close properly, allowing regurgitation to present.

Some solutions for treating mitral regurgitation in patients involve surgically stitching the edges of the native mitral valve leaflets directly to one another at or near a central portion of the leaflet edges, which may be referred to as an Alfieri procedure. Some solutions involve using a catheter delivery system to deliver a clip device to attempt to clip the edges of the leaflets together. However, the use of such clip devices can be associated with certain shortcomings when used to clip the middle of the leaflets where they overlap by about 2 mm or more. Furthermore, the use of multiple clips on the commissures of the mitral valve, where there may be more overlap of the leaflets, can result in a longer operation time and also joins the patient’s leaflets at the sides, restricting blood flow. Additionally, both the surgical and clip treatments can create undesirable stress on patient leaflets.

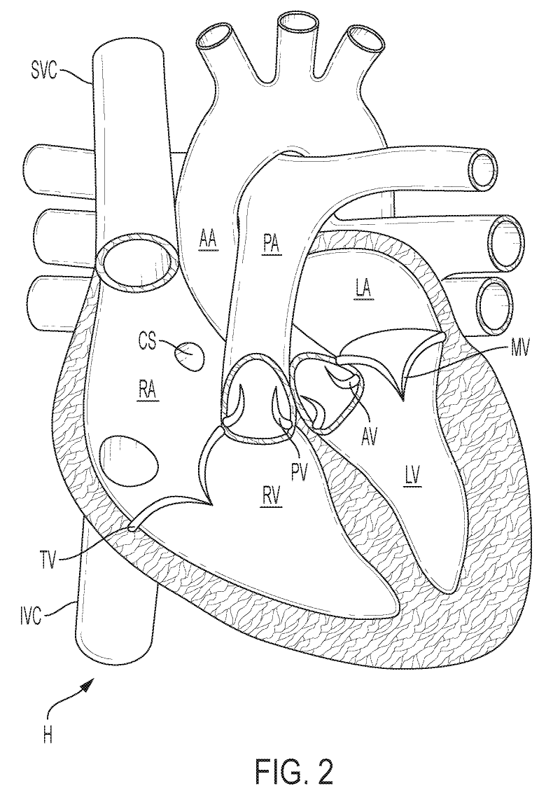

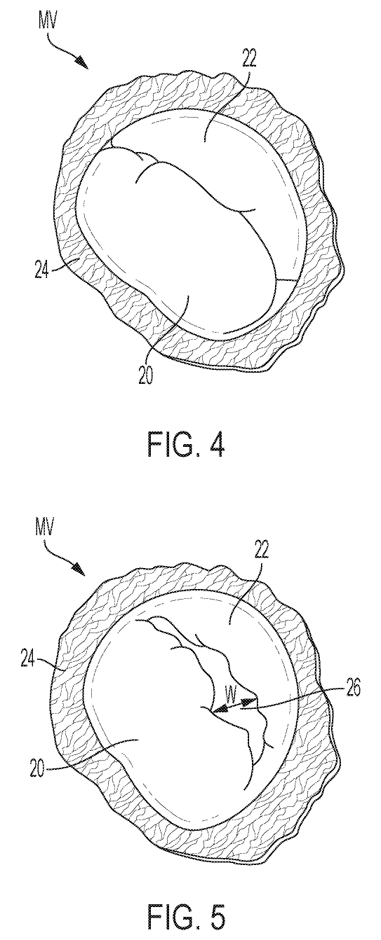

To provide further context for the inventive solutions disclosed herein, further details of the human cardiac anatomy are disclosed below with reference to FIGS. 1-6. FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 4 and 5) extending inward across the respective orifices that come together or “coapt” in the flowstream to form the one-way, fluid-occluding surfaces. The native valve repair systems of the present application are described primarily with respect to the mitral valve MV. Therefore, anatomical structures of the left atrium LA and left ventricle LV will be explained in greater detail. It should be understood that the devices described herein may also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA, and blood is collected in the left atrium from the pulmonary vein. In one exemplary embodiment, the systems and devices described by the present application are used to repair the function of a defective mitral valve MV. That is, the systems and devices are configured to help close the leaflets of the mitral valve to prevent blood from regurgitating from the left ventricle LV and back into the left atrium LA.

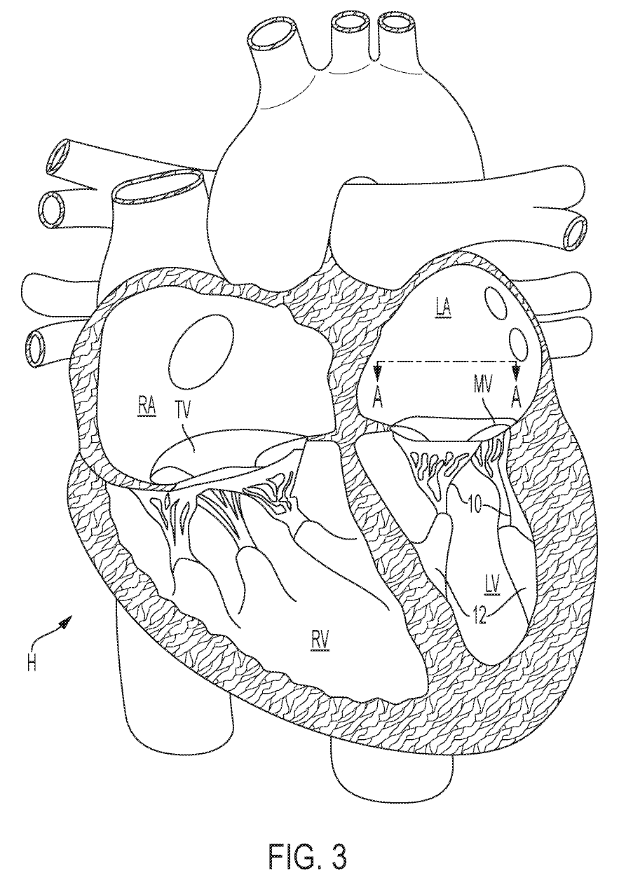

Referring now to FIGS. 1-6, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae 10. The chordae tendineae 10 are cord-like tendons that connect the papillary muscles 12 (i.e., the muscles located at the base of the chordae tendineae and within the walls of the left ventricle) to the leaflets 20, 22 of the mitral valve MV. The papillary muscles 12 serve to limit the movements of the mitral valve MV and prevent the mitral valve from being reverted. The mitral valve MV opens and closes in response to pressure changes in the left atrium LA and the left ventricle LV. The papillary muscles do not open or close the mitral valve MV. Rather, the papillary muscles brace the mitral valve MV against the high pressure needed to circulate blood throughout the body. Together the papillary muscles and the chordae tendineae are known as the subvalvular apparatus, which functions to keep the mitral valve MV from prolapsing into the left atrium LA when the mitral valve closes.

Various disease processes can impair proper function of one or more of the native valves of the heart H. These disease processes include degenerative processes (e.g., Barlow’s Disease, fibroelastic deficiency), inflammatory processes (e.g., Rheumatic Heart Disease), and infectious processes (e.g., endocarditis). In addition, damage to the left ventricle LV or the right ventricle RV from prior heart attacks (i.e., myocardial infarction secondary to coronary artery disease) or other heart diseases (e.g., cardiomyopathy) can distort a native valve’s geometry, which can cause the native valve to dysfunction. However, the vast majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in two different ways: (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow.

The second type of valve malfunction, valve regurgitation, occurs when the leaflets of the valve do not close completely thereby causing blood to leak back into the prior chamber (e.g., causing blood to leak from the left ventricle to the left atrium). There are three mechanisms by which a native valve becomes regurgitant—or incompetent—which include Carpentier’s type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier’s type II malfunction involves prolapse of one or more leaflets of a native valve above a plane of coaption. A Carpentier’s type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

Referring to FIG. 4, when a healthy mitral valve MV is in a closed position, the anterior leaflet 20 and the posterior leaflet 22 coapt, which prevents blood from leaking from the left ventricle LV to the left atrium LA. Referring to FIG. 5, regurgitation occurs when the anterior leaflet 20 or the posterior leaflet 22 of the mitral valve MV is displaced into the left atrium LA during systole. This failure to coapt causes a gap 26 between the anterior leaflet 20 and the posterior leaflet 22, which allows blood to flow back into the left atrium LA from the left ventricle LV during systole. As set forth above, there are several different ways that a leaflet (e.g. leaflets 20, 22 of mitral valve MV) may malfunction, which can thereby lead to regurgitation.

Referring to FIG. 5, in certain situations, the mitral valve MV of a patient can have a wide gap 26 between the anterior leaflet 20 and the posterior leaflet 22 when the mitral valve is in a closed position (i.e., during the systolic phase). For example, the gap 26 can have a width W between about 2.5 mm and about 17.5 mm, such as between about 5 mm and about 15 mm, such as between about 7.5 mm and about 12.5 mm, such as about 10 mm. In some situations, the gap 26 can have a width W greater than 15 mm. In any of the above-mentioned situations, a valve repair system is desired that is capable of engaging the anterior leaflet 20 and the posterior leaflet 22 to close the gap 26 and prevent regurgitation of blood through the mitral valve MV.

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) is primarily responsible for circulating the flow of blood throughout the body, malfunction of the mitral valve MV or the aortic valve AV is particularly problematic and often life threatening. Accordingly, because of the substantially higher pressures on the left side of the heart, dysfunction of the mitral valve MV or the aortic valve AV is much more problematic.

Malfunctioning native heart valves may either be repaired or replaced. Repair typically involves the preservation and correction of the patient’s native valve. Replacement typically involves replacing the patient’s native valve with a biological or mechanical substitute. Typically, the aortic valve AV and pulmonary valve PV are more prone to stenosis. Because stenotic damage sustained by the leaflets is irreversible, the most conventional treatments for a stenotic aortic valve or stenotic pulmonary valve are removal and replacement of the valve with a surgically implanted heart valve, or displacement of the valve with a transcatheter heart valve. The mitral valve MV and the tricuspid valve TV are more prone to deformation of leaflets, which, as described above, prevents the mitral valve or tricuspid valve from closing properly and allows for regurgitation or back flow of blood from the ventricle into the atrium (e.g., a deformed mitral valve MV may allow for regurgitation or back flow from the left ventricle LV to the left atrium LA). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae 10 becoming dysfunctional (e.g., the chordae tendineae may stretch or rupture), which allows the anterior leaflet 20 and the posterior leaflet 22 to be reverted such that blood is regurgitated into the left atrium LA. The problems occurring due to dysfunctional chordae tendineae 10 can be repaired by repairing the chordae tendineae or the structure of the mitral valve (e.g., by securing the leaflets 20, 22 at the affected portion of the mitral valve).

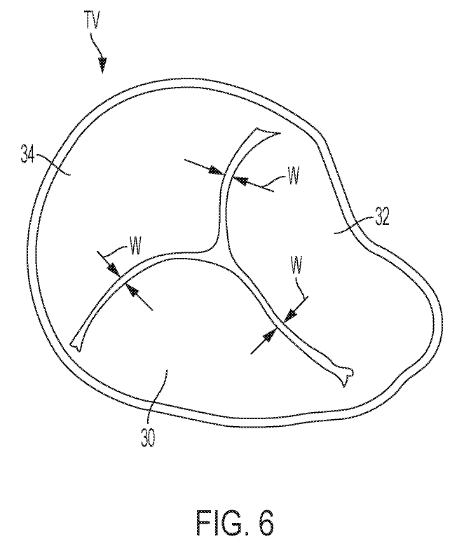

The systems and procedures disclosed herein refer to repairing the structure of a mitral valve. However, it should be understood that the systems, devices, and concepts provided herein can be used to repair any native valve, as well as any component of a native valve. Referring now to FIG. 6, any of the devices and concepts provided herein can be used to repair the tricuspid valve TV. For example, any of the systems, devices, methods, and concepts provided herein can be used between any two of the anterior leaflet 30, septal leaflet 32, and posterior leaflet 34 of the tricuspid valve TV to prevent regurgitation of blood from the right ventricle into the right atrium. In addition, any of the systems, devices, methods, and concepts provided herein can be used on all three of the leaflets 30, 32, 34 together to prevent regurgitation of blood from the right ventricle to the right atrium. That is, the valve repair systems provided herein can be centrally located between the three leaflets 30, 32, 34.

Some embodiments of systems for repairing a mitral or tricuspid valve in accordance with the present disclosure include a connector or an implantable prosthetic device. The connector and implantable prosthetic device can take a wide variety of different forms. For example, the implantable prosthetic device can be any device which holds a portion of the valve leaflets while the connector is implanted and that can be either removed (leaving the connector installed/implanted on the valve leaflets) or left installed/implanted on the native valve leaflets with the connector. For example, in some embodiments, an implantable prosthetic device can have an optional coaption element and at least one anchor for permanently or temporarily attaching to a valve leaflet. The optional coaption element can be configured to be positioned or disposed within the native heart valve orifice to at least partially fill the space between the valve leaflets or to form a more effective seal, thereby reducing or preventing regurgitation. The coaption element can be reduced in size or eliminated or omitted in certain embodiments disclosed herein, wherein the implantable prosthetic device may be removed after the connector is implanted. When included, the coaption element can have a structure that is impervious to blood and that allows the native leaflets to close around at least a portion of the coaption element during ventricular systole to block at least an amount of blood from flowing from the left or right ventricle back into the left or right atrium, respectively. The prosthetic device can be configured to seal against two or three native valve leaflets; that is, the device may be used in the native mitral (bicuspid) or tricuspid valves. The coaption element is sometimes referred to herein as a “spacer” because the coaption element can fill a space between improperly functioning native mitral or tricuspid leaflets that do not close/coapt completely.

The optional coaption element can have various shapes. In some embodiments, the coaption element can have an elongated cylindrical shape having an at least partially round cross-sectional shape. In some embodiments, the coaption element can have an oval cross-sectional shape, a crescent cross-sectional shape, or various other non-cylindrical shapes. When the prosthetic device is attached to the native valve leaflets, the coaption element can include an atrial portion positioned in or adjacent to the left atrium, a ventricular or lower portion positioned in or adjacent to the left ventricle, and a side surface that extends between the native mitral leaflets. In embodiments configured for use in the tricuspid valve, the atrial or upper portion can be positioned in or adjacent to the right atrium and the ventricular or lower portion can be positioned in or adjacent to the right ventricle, wherein the side surface extends between the native tricuspid leaflets.

The anchor component(s) of the prosthetic device can be configured to secure the device to one or both of the native mitral leaflets such that the coaption element is positioned between the two native leaflets. In embodiments configured for use in the tricuspid valve, the anchor can be configured to secure the device to one, two, or three of the tricuspid leaflets, such that the coaption element is positioned between two or three of the native leaflets. In some embodiments, the anchor can attach to the coaption element at a location adjacent to or associated with the ventricular portion of the coaption element. In some embodiments, the anchor can be coupled/attached to a shaft or actuation wire, to which the coaption element is also attached. In some embodiments, the anchor and the coaption element can be positioned independently with respect to each other by separately moving each of the anchor and the coaption element along the longitudinal axis of the shaft or actuation wire. In some embodiments, the anchor and the coaption element can be positioned simultaneously by moving the anchor and the coaption element together along the longitudinal axis of the shaft or actuation wire. The anchor can be configured to be positioned behind, at least in part, a native leaflet when implanted such that the leaflet is grasped by the anchor or pinned or captured between the anchor and the coaptation element.

The prosthetic device can be configured to be implanted via a delivery sheath/catheter. The optional coaption element and the anchor can be compressible to a radially compressed state and can be self-expandable to a radially expanded state when compressive pressure is released or when the various components are manually expanded. The prosthetic device can be configured for the anchor to be expanded radially away from the still-compressed coaption element initially in order to create a gap between the coaption element and the anchor. A native leaflet can then be positioned in the gap. The coaption element can be expanded radially, closing the gap between the coaption element and the anchor and capturing the leaflet between the coaption element and the anchor. In some embodiments, the anchor and coaption element are configured to self-expand. The implantation methods for various embodiments can be different and are more fully discussed below with respect to each embodiment. Additional information regarding these and other delivery methods can be found in U.S. Pat. No. 8,449,599 and U.S. Patent Application Publication Nos. 2014/0222136, and 2014/0067052, 2016/0331523 each of which is incorporated herein by reference in its entirety. U.S. Provisional Patent Application No. 62/744,031, filed October 10, 2018, entitled Heart Valve Sealing Devices and Delivery Devices Therefor, discloses an exemplary implantable prosthetic device, the disclosure of which is incorporated herein by reference in its entirety.

The disclosed prosthetic devices can be configured such that the anchor component is connected to one or more leaflets, thereby taking advantage of the tension from native chordae tendineae to resist high systolic pressure urging the device toward the left atrium. During diastole, the devices can rely on the compressive and retention forces exerted on the leaflet that is grasped by the anchor(s).

Deployable and releasable prosthetic devices, including but not limited to the disclosed deployable and releasable prosthetic devices, can be configured as part of a valve repair system that is used in conjunction with one or more connectors (e.g., sutures) that connect the leaflets of the heart valve together. The connectors can be configured in a variety of ways. For example, each of the one or more connectors may be a suture, a staple, a stitch/suture, or other suitable connector. The connectors may be separate from, or connected but separable from, the prosthetic device. The disclosed prosthetic device may be configured to grasp one or more leaflets of the heart valve to hold the heart valve (or portions of the heart valve, such as leaflets) in a position while the one or more connectors are being attached to the leaflets. Once the one or more connectors are in place and connecting the leaflets of the heart valve together, the implantable prosthetic device may be kept in position and used in combination with the one or more connectors, or the implantable prosthetic device may be released and removed from the heart while the connectors remain in position.

The terms “suture” and “stitch” are used herein according to their broad/plain and ordinary meaning and may refer to any elongate cord strip, strand, line, rope, wire, filament, tie, string, ribbon, strap, or portion thereof, or other type/form of material used in medical procedures (e.g., ePTFE suture, for example, GORE-TEX® sutures, W.L. Gore, Newark, Delaware). Furthermore, embodiments of the present disclosure may be implemented in connection with non-surgical or non-biological suture or stich implantation. With respect to the present disclosure, one having ordinary skill in the art will understand that a wire, staple, or other similar material may be used in place of a suture/stitch. Furthermore, in some contexts herein, the terms “stitch” and “suture” may be used substantially interchangeably. In addition, use of the singular form of any of the suture-related terms listed above, including the terms “suture” and “stitch,” may be used to refer to a single suture/cord, or to a portion thereof. Furthermore, sutures/stitches may be used with a pledget to reduce tissue damage or spread the suture or stitch load over a broader surface area.

The implantation methods for various embodiments of the disclosed prosthetic devices and the configuration of the disclosed prosthetic devices can be adapted to both allow for deployment and, in some cases, release of the prosthetic devices as well as the deployment and attachment of the one or more connectors to the leaflets of the heart valve after deployment of the prosthetic devices.

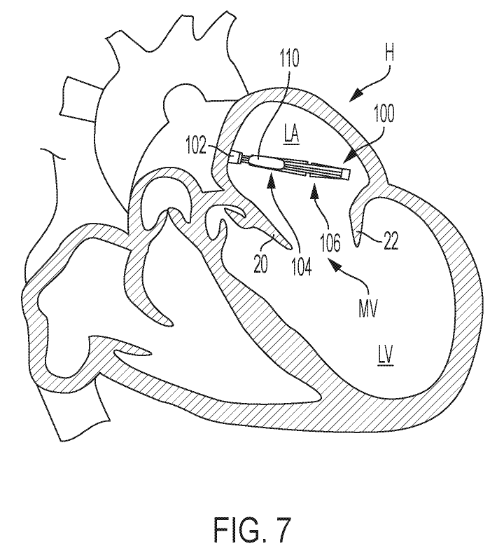

Referring now to FIGS. 7-12, an exemplary embodiment of an implantable prosthetic device 100 is shown being delivered and implanted within the native mitral valve MV of the heart H. As with the implantable device disclosed in detail in U.S. Provisional Patent Application No. 62/744,031, the implantable prosthetic device 100 may be deployed from a delivery sheath or means for delivery 102 and optionally includes a coaption portion 104 and an anchor portion 106. The coaption portion/element is optional or may be minimized in size in the present application, since the prosthetic device may be removed, and thus the coaptation portion would not serve to improve leaflet coaptation postoperatively and the utility thereof is reduced compared to implementations in which the prosthetic device remains implanted in the patient post-operatively. The coaption portion 104 of the implantable prosthetic device 100 includes a coaption element or means for coapting 110 that is adapted to be implanted between the leaflets of the native mitral valve and may be slidably attached to an actuation wire or shaft 112 during one or more periods of the process.

The prosthetic device 100 may be considered a leaflet-securing implant due to its ability to clamp, grip, hold, clip, pinch, or otherwise secure portions of two or more leaflets thereto, thereby maintaining a fixed relative position of such leaflet portions. The device 100 may be a clip device or may include a central spacer element or portion in some embodiments, while other embodiments may omit such features. The prosthetic device 100 may further be considered an edge-to-edge repair device in that it is configured to attach to or between the edges of the target valve leaflets or repair or compensate for a gap that may undesirably form or exist between the edges of the native valve leaflets.

In some embodiments, the overall shape of the coaption element or means for coapting 110 is an elliptical or oval cross section when seen from the surgeon’s view (top view), a tapered shape or cross section when seen from an LVOT view (side view), and a substantially round shape or rounded shape when seen from an intra-commissural view. In some embodiments, a blend of these three geometries can result in the three-dimensional shape of the illustrated coaption element or means for coapting 110.

The anchor portion 106 can be actuatable between open and closed conditions/configurations and can take a wide variety of forms, such as, for example, paddles, gripping elements, or the like. Actuation of the actuation wire or means for actuating 112 can open or close the anchor portion 106 of the implantable prosthetic device 100 to grasp the mitral valve leaflets during implantation.

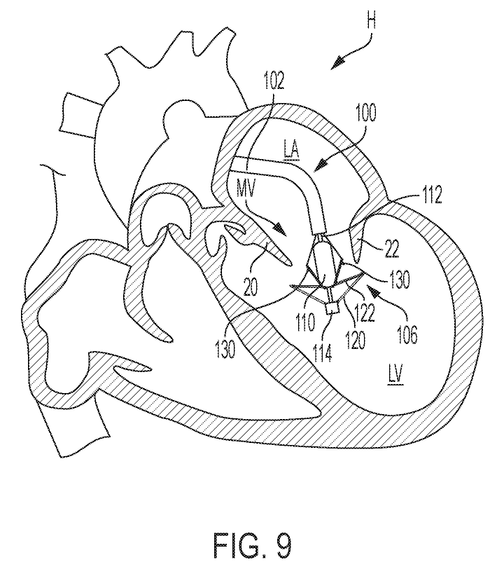

Referring to FIG. 9, in the illustrated embodiment, the anchor portion 106 of the implantable prosthetic device 100 includes outer paddles 120 and inner paddles 122 that may be connected between a cap 114 and the distal end of the coaption element or means for coapting 110. The anchor portion 106 further includes attachment portions or gripping members. The illustrated gripping members can comprise barbed clasps 130 that can be opened separately by pulling on an attached actuation line 116 that extends through the delivery sheath or means for delivery 102 to the barbed clasp 130.

The coaption element or means for coapting 110 and paddles 120, 122 can be formed from a flexible material that may be a metal fabric, such as a mesh, woven, braided, or formed in any other suitable way or a laser cut or otherwise cut flexible material. The flexible material may be cloth, shape-memory alloy wire—such as Nitinol—to provide shape-setting capability, or any other flexible material suitable for implantation in the human body.

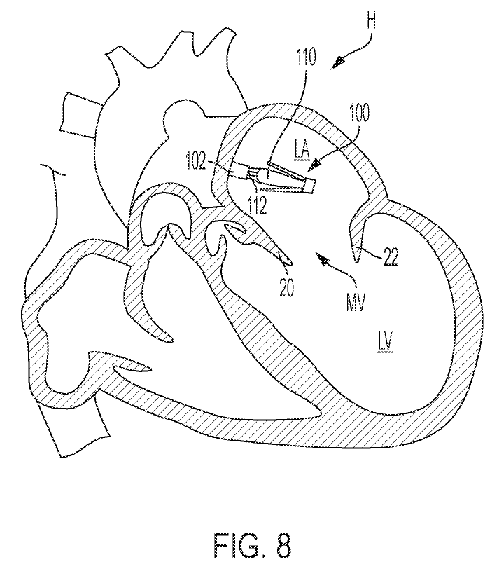

Referring now to FIG. 7, the delivery sheath or means for delivery 102 (e.g., catheter delivery system) is inserted into the left atrium LA, for example, through the septum, and the implantable prosthetic device 100 is deployed from the delivery sheath or means for delivery 102 in an open (e.g., fully open) condition/configuration. The actuation wire or means for actuating 112 can then be retracted to move the implantable prosthetic device 100 into an at least partially closed (e.g., fully closed) condition/configuration, as shown in FIG. 8.

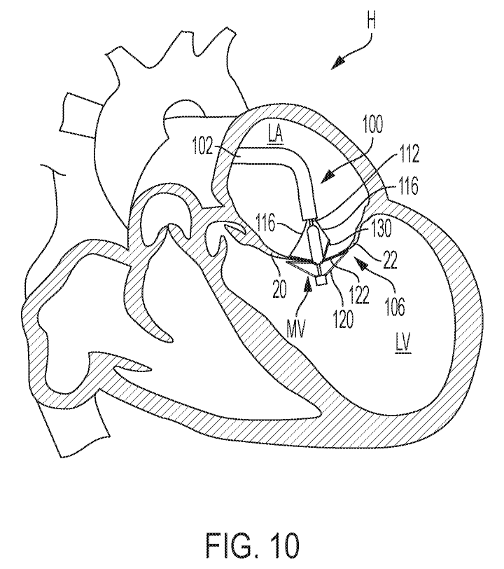

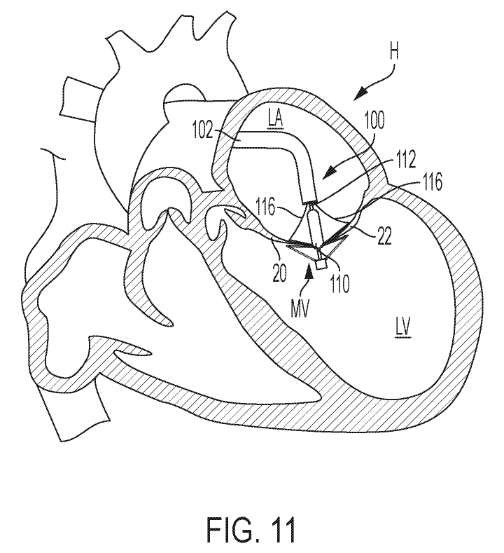

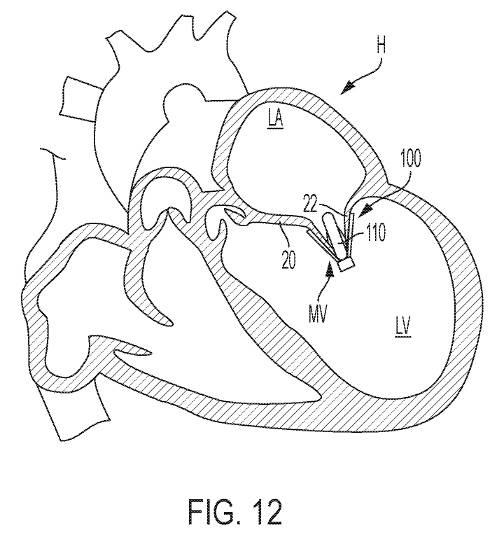

As can be seen in FIG. 9, the implantable prosthetic device 100 can be moved into a position at least partially within the mitral valve MV, into the ventricle LV, and at least partially opened so that the leaflets 20, 22 can be grasped. Referring now to FIG. 10, an actuation line 116 can be extended to close one of the clasps 130, capturing the valve leaflet 20. FIG. 11 shows another actuation line 116 being extended to close the other clasp 130, capturing the remaining leaflet 22. As can be seen in FIG. 12, one or more of the delivery sheath or means for delivery 102, the actuation wire or means for actuating 112, and the actuation line(s) 116 can then be retracted, in some implementations, and the implantable prosthetic device 100 can be left in place fully closed and deployed in the native mitral valve MV.

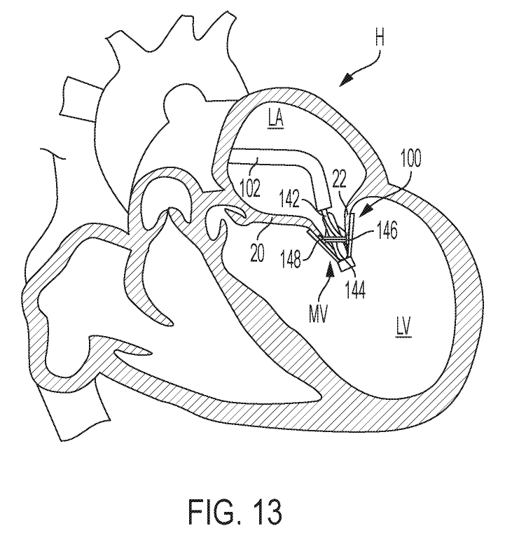

As shown in FIGS. 13-18, the implantable prosthetic device 100 can facilitate one or more connectors (e.g., sutures) 140 being deployed and implanted to connect the two valve leaflets 20, 22 together while the implantable prosthetic device 100 is in place. The one or more connectors 140 can be attached to the leaflets 20, 22 and left in place along with the fully closed and implantable prosthetic device 100 or the one or more connectors 140 can be attached to the leaflets 20, 22 and left in place and the implantable prosthetic device 100 can be released and removed along with the delivery sheath or means for delivery 102, the actuation wire or means for actuating 112, or the actuation line(s) 116. The connector 140 can be considered an edge-to-edge valve repair device because it is configured to be coupled to or approximate the edges of two or more leaflets to one another to thereby reduce a gap that may otherwise present/exist between the edges of the native valve leaflets.

Referring to FIG. 13, in the illustrated embodiment, the clasps 130 on the implantable prosthetic device 100 have been closed to capture the valve leaflets 20, 22, respectively, while the delivery sheath or means for delivery 102 or the actuation wire or means for actuating 112 have not been retracted. In some implementations, a secondary delivery sheath or secondary means for deliver (e.g., catheter delivery system) 142 may have been deployed through the delivery sheath or means for delivery 102 to deploy the one or more connectors 140. In some embodiments, the secondary delivery sheath or secondary means for delivery 142 includes an open distal end 144 and may be steerable such that the distal end 144 can be positioned as desired, such as adjacent a valve leaflet 20, 22 as shown in FIG. 13.

The one or more connectors 140 can be deployed through the secondary delivery sheath or secondary means for delivery 142 (or through any other delivery system or mechanism). The one or more connectors 140 can be configured in a variety of ways. Any suitable connector or connecting means 140 capable of being deployed through a delivery sheath or a catheter and able to connect the valve leaflets 20, 22 together may be used. For example, the connector or connecting means 140 may be a suture, a pledgeted suture, a staple, a stitch, a wire, or other suitable connecting device.

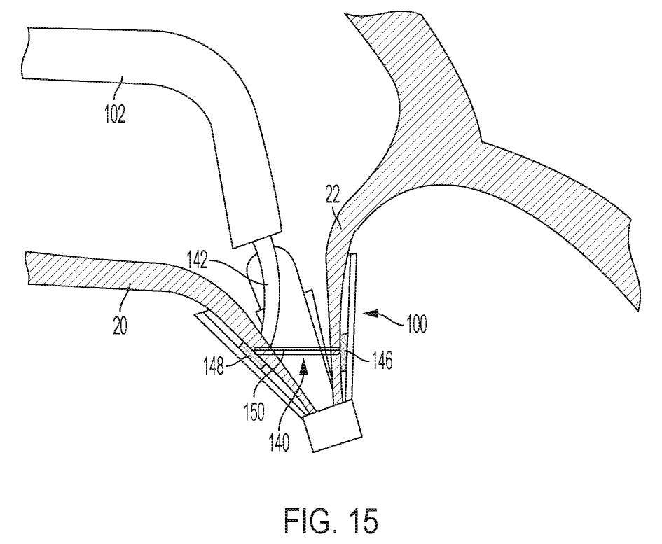

As best illustrated in FIG. 15, in the illustrated embodiment, the connector 140 includes a first tissue anchor 146 positioned on the ventricular side of the second valve leaflet 22 and a second tissue anchor 148 positioned on the ventricular side of the first valve leaflet 20. The first anchor 146 and the second anchor 148 are connected to each other, through the leaflets 20, 22, by a connecting portion. In the illustrated embodiment, the connecting portion is a suture line 150.

Referring to FIG. 13, with the distal end 144 of the secondary delivery sheath or secondary means for delivery 142 positioned adjacent the atrial side of the second valve leaflet 22, the second valve leaflet 22 may be pierced to create an opening (not shown) therein. The second valve leaflet 22 may be pierced in any of a variety of ways. For example, a piercing device (not shown), such as a needle or other pointed-tip or sharp-edged device may be delivered through the secondary delivery sheath or secondary means for delivery 142 to the distal end 144 and extended or pushed through the second valve leaflet 22. Alternatively, the distal end 144 of the secondary delivery sheath or secondary means for delivery 142 may be adapted to pierce the valve leaflet 22. For example, the distal end 144 of the delivery sheath may be sharp or pointed, like a needle, but may also include a lumen for delivering the catheter.

Once the second valve leaflet 22 has been pierced, the connector 140 may be delivered through the secondary delivery sheath or secondary means for delivery 142 to the distal end 144 and extended or pushed through the opening in the second valve leaflet 22. In the illustrated embodiment, the first anchor 146 is attached to, or formed at, the end of the suture line 150. The first anchor may be attached to, or formed at, the end of the suture line 150 while both the suture line 150 and the first anchor 146 are delivered through the secondary delivery sheath or secondary means for delivery 142. In other embodiments, the first anchor 146 may be attached to, or formed in, the end of the suture line 150 after the suture line 150 is delivered through the secondary delivery sheath or secondary means for delivery 142.

The first anchor 146 may be configured in a variety of ways. Any configuration that can be positioned adjacent the second valve leaflet 22 to secure the suture line 150 to the second valve leaflet 22 may be used. For example, the first anchor 146 can be a pledget, a knot formed in the suture line 150, a stop, or other suture line anchoring device. The first anchor 146 may be collapsible/expandable such that within the secondary delivery sheath or secondary means for delivery 142, the first anchor 146 is in a collapsed state. Once the first anchor 146 exits the distal end 144 of the secondary delivery sheath or secondary means for delivery 142, the first anchor 146 may expand. In one exemplary embodiment, the first anchor 146 includes a shape-memory alloy wire—such as Nitinol—to provide shape-setting capability. In the collapsed state, the first anchor 146 may be received through the opening in the second valve leaflet 22. In the expanded state, the first anchor 146 can have a shape and size that does not fit through the opening in the second valve leaflet 22. Although described in certain contexts herein as comprising pledgets or other structures, the anchors 146, 148 may be formed at least in part of bulky suture knots having a width dimension sufficient to prevent the knot/anchor from being drawn back through the opening in the leaflet. Such knot(s) can be formed from a unitary suture with the suture line 150. The connector anchors 146, 148 may be disposed between the clamps/anchors of the prosthetic device 100 and the respective leaflets prior to removal of the prosthetic device 100.

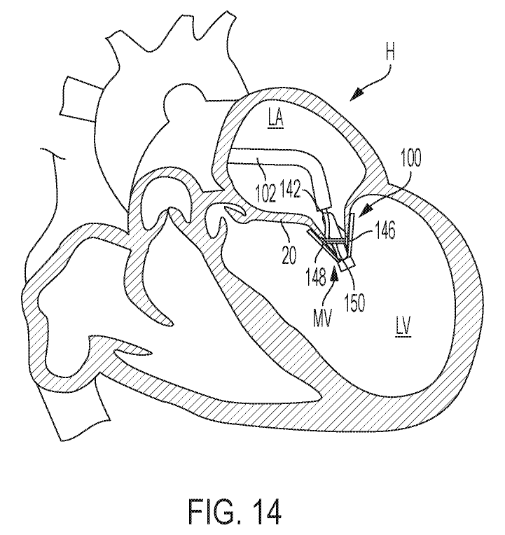

Referring to FIGS. 14 and 15, once the connector 140 is installed onto the second valve leaflet 22, the secondary delivery sheath or secondary means for delivery 142 may be steered such that the distal end 144 is positioned adjacent the atrial side of the first valve leaflet 20 and the suture line 150 extends between the leaflets 20, 22.

Once the distal end 144 is adjacent the atrial side of the first valve leaflet 20, the first valve leaflet 20 may be pierced in the same manner as described regarding the second valve leaflet 22. Once the first valve leaflet 20 has been pierced, the suture line 150 may be extended through the opening formed in the first valve leaflet 20 and the second anchor 148 may be attached to or formed in the suture line 150 adjacent the second valve leaflet 22 to secure the suture line 150 to the first valve leaflet 20. The second anchor 148 can be substantially similar to the first anchor 146 and may be deployed or formed in the same manner as the first anchor 146. In other embodiments, however, the first anchor 146 and the second anchor 148 may be different.

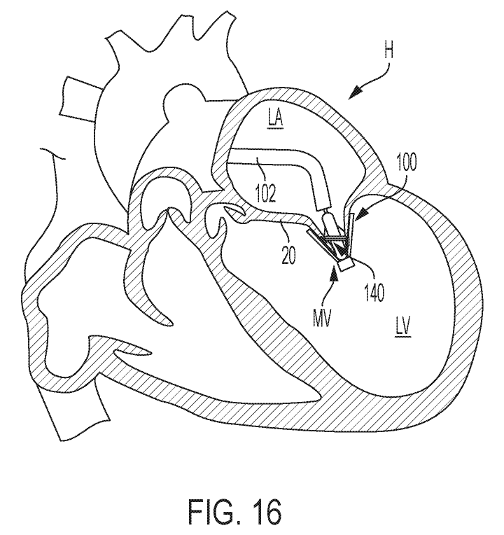

Referring to FIG. 16, with the connector(s) 140 in place, the first anchor 146 and the second anchor 148 can be positioned against the ventricular side of the second valve leaflet 22 and first valve leaflet 20, respectively. The suture line 150 extends from the first anchor 146 to the second anchor 148 through both of the valve leaflets 20, 22. In this way, the connector 140 can hold the valve leaflets 20, 22 together in the area of the connector 140. In some embodiments, the remaining suture line 150 extending from the second anchor 148 up through the secondary delivery sheath or secondary means for delivery 142 can be cut to detach the secondary delivery sheath or secondary means for delivery 142 from the connector 140 and the secondary delivery sheath or secondary means for delivery 142 can be withdrawn.

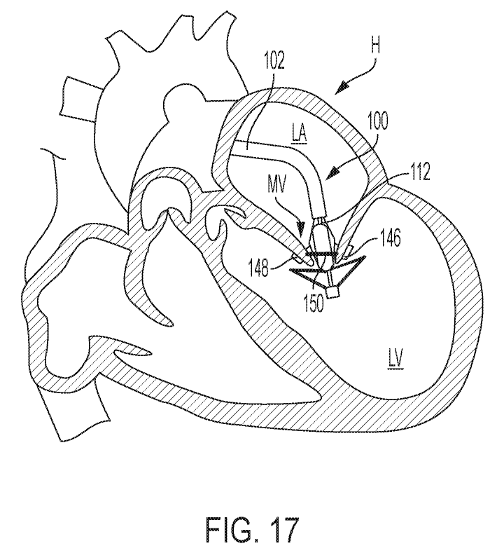

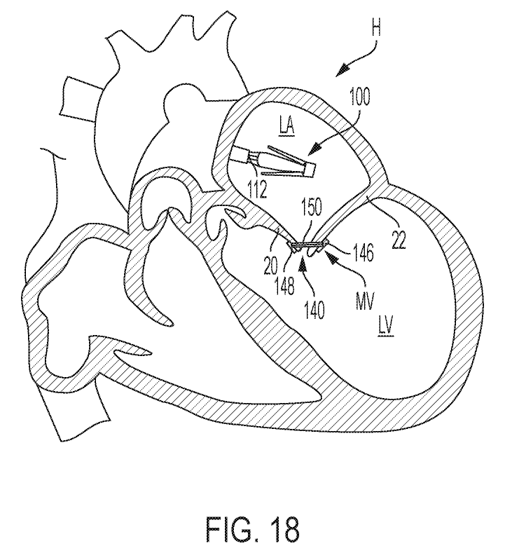

Referring to FIG. 17-18, with the connector 140 in place, the implantable prosthetic device 100 can be kept in place and work in conjunction with the one or more connectors 140, or, as illustrated, can be removed such that the one or more connectors 140 holds the valve leaflets together without the ongoing assistance of the implantable prosthetic device 100.

To remove the implantable prosthetic device 100, the actuation lines 116 can be retracted to open the clasps 130 and release each of the valve leaflet 20, 22. The actuation wire or means for actuating 112 can then be extended to move the implantable prosthetic device 100 into the fully open condition shown in FIG. 7 or another position that allows the prosthetic device 100 to be removed. The implantable prosthetic device 100 and the delivery sheath or means for delivery 102 are then retracted from the left atrium LA through the septum.

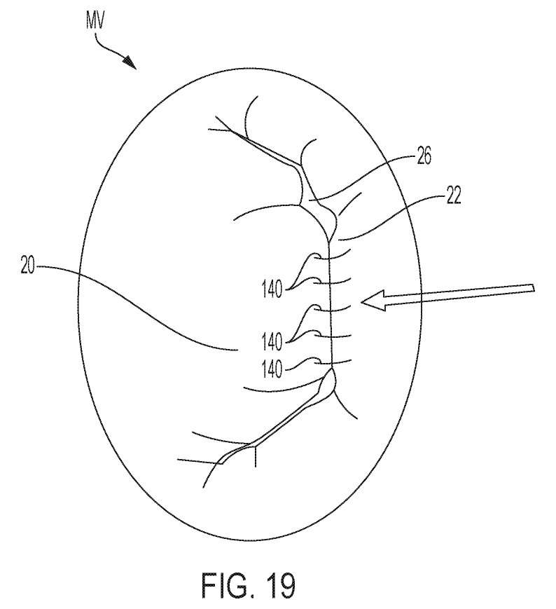

Referring to FIG. 19, the atrial side of the mitral valve MV is shown with a plurality of connectors 140 installed. In particular, five separate connectors 140, in the form of anchored sutures, extend from the first valve leaflet 20 to the second valve leaflet 22 to close the gap 26 and prevent regurgitation of blood through the mitral valve MV. In the illustrated embodiment, the five connectors 140 are evenly spaced from each other and parallel. In other embodiments, however, the number of connectors 140 can be greater than five or less than five and the connectors 140 may not be parallel to each other or evenly spaced.

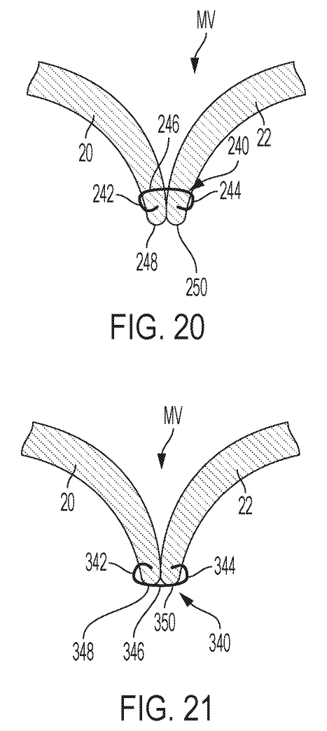

Referring to FIG. 20, another exemplary embodiment of a connector 240 is shown installed in the first valve leaflet 20 and second valve leaflet 22 of the mitral valve MV. The connector 240 may be configured in a variety of ways. In the illustrated embodiment, the connector 240 is formed as a staple or wire having a first end 242 connected to an opposite a second end 244 by an intermediate connecting portion 246. The connecting portion 246 extends from the ventricular side of the first valve leaflet 20 through both the first valve leaflet 20 and the second valve leaflet 22 to the ventricular side of the second valve leaflet 22. Thus, the connecting portion 246 serves to connect the first valve leaflet 20 and the second valve leaflet 22.

The first end 242 and the second end 244 serve as anchors to attach or anchor the connector 240 to the first valve leaflet 20 and the second valve leaflet 22. As shown in FIG. 16, both the first end 242 and the second end 244 can be curved or curled downward toward the tips 248, 250 of the first valve leaflet 20 and the second valve leaflet 22 (i.e. toward the left ventricle) and inward into the first valve leaflet 20 and the second valve leaflet 22. Thus, the first end 242 and the second end 244 of the connector 240 may pierce into the first valve leaflet 20 and the second valve leaflet 22, respectively, to secure the connector 240 to the leaflets 20, 22. In the illustrated embodiment, the first end 242 and the second end 244 of the connector 240 are imbedded into the first valve leaflet 20 and the second valve leaflet 22, respectively.

The connector 240 may be made of any suitable material or materials. In one exemplary embodiment, the connector 240 is made from, or includes, a shape-memory alloy wire—such as Nitinol—to provide shape-setting capability. In a free state, the illustrated connector 240 has the shape illustrated in FIG. 20. In other embodiments, however, the connector 240 may be shaped differently. Any shape that allows the connector 240 to anchor into and connect the leaflets 20, 22 may be used.

The use of shape-memory alloy materials can allow the connector 240 to be delivered and deployed through, for example, the secondary delivery sheath or secondary means for delivery 142 discussed in relation to the embodiment of FIGS. 13-18. Thus, the connector 240, within the secondary delivery sheath or secondary means for delivery 142, may be in a generally elongated configuration. For example, the open distal end 144 of the secondary delivery sheath or secondary means for delivery 142 may be steered such that the distal end 144 is positioned adjacent the atrial side of the first valve leaflet 20. A piercing device (not shown) may be delivered through the secondary delivery sheath or secondary means for delivery 142 to the distal end 144 and extended or pushed through the first valve leaflet 20 to create an opening in the first valve leaflet 20. Alternatively, the first end 242 of the connector 240 or the distal end 144 may be configured as a piercing device and used to create an opening in the first valve leaflet 20.

Once the first valve leaflet 20 has been pierced, the connector 240 may be delivered through the secondary delivery sheath or secondary means for delivery 142 to the distal end 144 and extended or pushed through the opening in the first valve leaflet 20. Once the first end 242 of the connector 240 exits the distal end 144, the shape-setting capability of the material of the connector 240 cause the first end 242 to revert to its free state. In the illustrated embodiment, the first end 242 curls downward and inward to embed into the ventricular side of the first valve leaflet 20.

Once the connector 240 is installed onto the first valve leaflet 20, the secondary delivery sheath or secondary means for delivery 142 may be steered such that the distal end 144 is positioned adjacent the atrial side of the second valve leaflet 22. The second valve leaflet 22 may be pierced in the same manner as described regarding the first valve leaflet 20.

Once the second valve leaflet 22 has been pierced, the second end 244 of the connector 240 may be delivered extended or pushed through the opening in the second valve leaflet 22. Once the second end 244 of the connector 240 exits the distal end 144, the shape-setting capability of the material of the connector 240 causes the second end 244 to revert to its free state. In the illustrated embodiment, the second end 244 curls downward and inward to embed into the ventricular side of the second valve leaflet 22. Thus, the first end 242 is anchored in the first valve leaflet 20, the second end 244 is anchored in the second valve leaflet 22, and the connecting portion 246 connects the first valve leaflet 20 and the second valve leaflet 22.

In the illustrated embodiment, the connecting portion 246 is slightly curved. In other embodiments, however, the connecting portion 246 can be any suitable shape, such as straight, less curvature, or more curvature.

It will be understood that, similar to the connectors 140 shown in FIG. 9, more than one connector 240 may be used to extend from the first valve leaflet 20 to the second valve leaflet 22 to close the gap 26 and prevent regurgitation of blood through the mitral valve MV. Furthermore, the connector 246 may be implanted while a prosthetic leaflet-holding device, such as similar to the device 100 described above or other clip or clamp device, holds the leaflets together. That is, any of the connectors described in any of the embodiments in FIGS. 20-26 may be deployed with a prosthetic leaflet-holding device in place to facilitate the placement of the connector(s). For clarity and convenience, suture-holding prosthetic devices are not shown in FIGS. 20-26 allow for greater detail of illustration with respect to the respective connector(s) shown.

Referring to FIG. 21, another exemplary embodiment of a connector 340 is shown installed in the first valve leaflet 20 and second valve leaflet 22 of the mitral valve MV. The connector 340 is substantially similar to the connector 240 in that it is formed as a staple or wire having a first end 342 connected to an opposite a second end 344 by a connecting portion 346. The first end 342 and the second end 344 serve as anchors to attach or anchor the connector 340 to the first valve leaflet 20 and the second valve leaflet 22, respectively. As with the connector 240, the connector 340 may be formed from or include a shape-memory alloy wire—such as Nitinol—to provide shape-setting capability.

As shown in FIG. 21, when installed however, the first end 342 and the second end 344 of connector 340 curve or curl upward toward the base of the first valve leaflet 20 and the second valve leaflet 22 (i.e. toward the left atrium) and inward into the first valve leaflet 20 and the second valve leaflet 22. In addition, the connecting portion 346 does not extend through openings in the valve leaflets 20, 22. Instead, the connecting portion 346 extends around the tips 348, 350 of the valve leaflets 20, 22, respectively.

During installation, instead of positioning the distal end 144 of the secondary delivery sheath or secondary means for delivery 142 adjacent the atrial side of the valve leaflets 20, 22 in order to pierce the leaflets 20, 22, the distal end 144 is steered to the ventricular side of the valve leaflets 20, 22. In particular, the secondary delivery sheath or secondary means for delivery 142 may be extended into the left ventricle and steered such that the distal end 144 is adjacent the ventricular side of the first valve leaflet 20. The connector 340 may be delivered through the secondary delivery sheath or secondary means for delivery 142 and out of the distal end 144. Once the first end 342 of the connector 340 exits the distal end 144, the shape-setting capability of the material of the connector 340 cause the first end 342 to revert to its free state. In the illustrated embodiment, the first end 342 curls upward and inward to embed into the ventricular side of the first valve leaflet 20.

Once the connector 340 is installed onto the first valve leaflet 20, the secondary delivery sheath or secondary means for delivery 142 may be steered such that the distal end 144 is positioned adjacent the ventricular side of the second valve leaflet 22 and the second end 344 of the connector 340 may be extended or pushed out of the distal end 144. Once the second end 344 exits the distal end 144, the shape-setting capability of the material of the connector 340 cause the second end 344 to revert to its free state. In the illustrated embodiment, the second end 344 curls upward and inward to embed into the ventricular side of the second valve leaflet 22. Thus, the first end 342 is anchored in the first valve leaflet 20, the second end 344 is anchored in the second valve leaflet 22, and the connecting portion 246 extends around the valve leaflet tips 348, 350 to connect the first valve leaflet 20 and the second valve leaflet 22.

In the illustrated embodiment, the connecting portion 346 is slightly curved. In other embodiments, however, the connecting portion 346 can be any suitable shape, such as straight, less curvature, or more curvature.

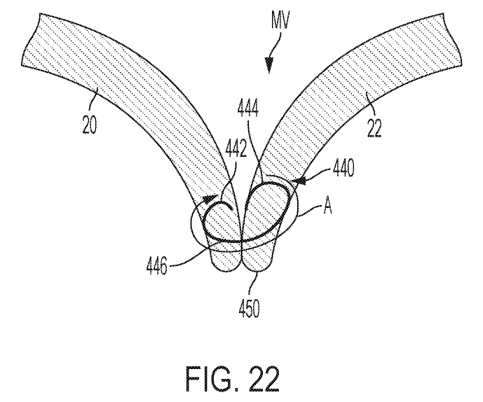

Referring to FIG. 22, another exemplary embodiment of a connector 440 is shown installed in the first valve leaflet 20 and second valve leaflet 22 of the mitral valve MV. The connector 440 is similar to the connector 340 in that it is formed as a staple or wire having a first end 442 connected to an opposite a second end 444 by a connecting portion 446. The first end 442 and the second end 344 serve as anchors to attach or anchor the connector 440 to the first valve leaflet 20 and the second valve leaflet 22, respectively. As with the connector 240, the connector 440 may be formed from or include a shape-memory alloy wire—such as Nitinol—to provide shape-setting capability. In the illustrated embodiment, the connector 440 has a continuous curled shape.

During installation, the open distal end 144 of the secondary delivery sheath or secondary means for delivery 142 may be steered such that the distal end 144 is positioned adjacent the atrial side of the second valve leaflet 22. The first end 342 of the connector 240 may be configured as a piercing device and used to create an opening in the second valve leaflet 22.

The connector 440 may be delivered through the secondary delivery sheath or secondary means for delivery 142 to the distal end 144 and extended or pushed into the first valve leaflet 20. Once the first end 442 of the connector 440 exits the distal end 144, the shape-setting capability of the material of the connector 240 cause the portion of the connector 440 deployed from the secondary delivery sheath or secondary means for delivery 142 to revert to its free state. In the illustrated embodiment, the first end 442 of the connector 440 follows the curved path shown by arrow A as the connector 440 deployed from the secondary delivery sheath or secondary means for delivery 142. In particular, the first end 442 extends through the second valve leaflet 22 in a downward curved path before exiting the atrial side of the second valve leaflet 22 at a location closer to the tip 450 of the second valve leaflet 22 than the location where the first end 442 initially entered the second valve leaflet 22. After exiting the atrial side of the second valve leaflet 22, the first end enters the atrial side of the first valve leaflet 20 and extends in an upward curved path until the second end 444 exits the distal end 144 of the secondary delivery sheath or secondary means for delivery 142 (i.e., the connector 440 is fully installed).

In the illustrated embodiment, the connector 440 is not exposed on the ventricular sides of the first and second valve leaflets 20, 22. In other embodiments, however, as the first end 442 tunneled the curved path through the first and second valve leaflets 20, 22, the first end 442 may exit and reenter the ventricular sides of the first and second valve leaflets 20, 22. Thus, a portion of the embedded connector 440 may be exposed on the ventricular sides of the first and second valve leaflets 20, 22

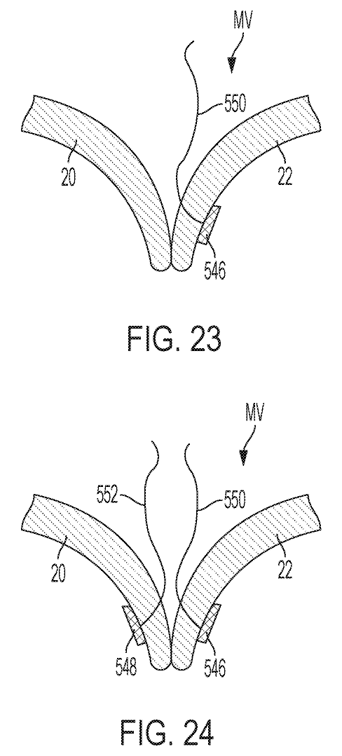

Referring to FIGS. 23-26, another exemplary embodiment of a connector 540 is shown being installed in the first valve leaflet 20 and second valve leaflet 22 of the mitral valve MV. The connector 540 is similar to the connector 140 of FIGS. 13-18 in that the connector 540 includes a first anchor 546 positioned on the ventricular side of the second valve leaflet 22 for anchoring a suture line to the second valve leaflet 22 and a second anchor 548 positioned on the ventricular side of the first valve leaflet 20 for anchoring a suture line to the first valve leaflet 20.

However, while the connector 140 has a suture line 150 that extends between the first anchor 146 and the second anchor 148, the connector 540 has a first suture line 550 attached to the first anchor 546 and a separate, second suture line 552 attached to the second anchor 548. The connector 540 also includes a suture-locking device 554 configured to capture first suture line 550 and second suture line 552 and lock them in position relative to the suture-locking device 554.

The connector 540 can be delivered and deployed through, for example, one or more delivery sheathes or means for delivery in a similar manner as discussed in relation to the embodiment of FIGS. 13-18. The first anchor 546 and first suture line 550 may be deployed via the same delivery sheath or means for delivery as the second anchor 548 and second suture line 552 or may be deployed via a different delivery sheath or secondary means for delivery.

As shown in FIGS. 23-24, the second valve leaflet 22 may be pierced and the first anchor 546 may be positioned on the ventricular side of the second valve leaflet 22 with the first suture line 550 extending through the second valve leaflet 22. Similarly, the first valve leaflet 20 may be pierced and the second anchor 548 may be positioned on the ventricular side of the first valve leaflet 20 with the second suture line 552 extending through the first valve leaflet 20.

The suture-locking device 554 can be deployed via the delivery sheath or means for delivery 102. The suture-locking device 554 may be mounted onto the first and second suture lines 550, 552 such that the first and second suture lines 550, 552 extend through the suture-locking device 554. The suture-locking device 554 may be mounted onto the first and second suture lines 550, 552 prior to the suture-locking device 554 being deployed via the delivery sheath or means for delivery 102 or in the left atrium, after the suture-locking device 554 exits the delivery sheath or means for delivery 102.

The suture-locking device 554 may be configured in a variety of ways. Any device that can facilitate drawing the first and second valve leaflets 20, 22 together and locking the first and second suture lines 550, 552 in place in order to hold the first and second valve leaflets 20, 22 together may be used. In the illustrated embodiment, the suture-locking device includes a wedge-type device 556 and a locking nut/clip 558.

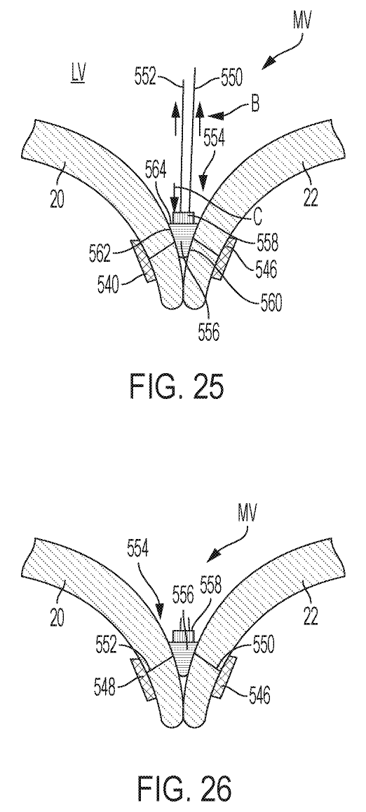

Referring to FIG. 25, the wedge 556 includes a first angled engagement surface 560 having an channel (not shown) that receives the first suture line 550 and a second angled engagement surface 562, opposite the first angled engagement surface 560, having a second channel (not shown) that receives the second suture line 552. The first angled engagement surface 560 and the second angled engagement surface 562 form the tapering wedge shape of the wedge 556 and are connected via a base surface 564. The first suture line 550 and the second suture line 552 exit the wedge 556 via a locking aperture (not shown) in the base surface 564.

The locking nut 558 is configured to, at least partially, be received in the locking aperture. The locking nut 558 is annular and includes an internal, longitudinal passage (not shown) through which the first suture line 550 and the second suture line 552 extend. The locking nut 558 can be configured to attach to the wedge 556 in a manner which locks the first suture line 550 and the second suture line 552 in place relative to the wedge 556. In an exemplary embodiment, the locking nut 558 includes a male threaded portion (not shown) configured to threadably engage a corresponding female threaded portion (not shown) in the locking aperture of the wedge 556. In some embodiments, the locking nut 558 comprises a clip or other form

Referring to FIG. 25, when the suture-locking device is positioned such that the first suture line 550 and the second suture line 552 are received through the wedge 556, the first suture line 550 and the second suture line 552 can be pulled in the direction of the left ventricle LV as shown by arrows B in FIG. 25 and the wedge 556 can be pushed in the opposite direction as shown by arrow C. As a result, the first valve leaflet 20 and the second valve leaflet 22 are drawn together such that the wedge 556 is sandwiched between the first valve leaflet 20, which engages the second angled engagement surface 562, and the second valve leaflet 22, which engages the second angled engagement surface 562.

To lock the first valve leaflet 20 and the second valve leaflet 22 together, the locking nut 558 can be attached to the wedge 556 to capture the first suture line 550 and the second suture line 552 between the locking nut 558 and the wedge 556. For example, the locking nut 558 can be threaded into the lacking aperture until the first suture line 550 and the second suture line 552 are sandwiched between the locking nut 558 and an interior surface of the wedge 556.

Once the first suture line 550 and the second suture line 552 are captured between the locking nut 558 and the wedge 556, the first suture line 550 and the second suture line 552 can be cut external to the locking nut 558.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

What is claimed is:

1. A method of treating a heart valve comprising:
inserting a catheter delivery system into a heart;
deploying a device from the catheter delivery system into the heart, the device comprising a plurality of paddles;
moving the plurality of paddles into a partially open position;
moving the device such that the plurality of paddles engage leaflets of the heart valve;
piercing a first one of the leaflets of the heart valve;
moving a first anchor and a portion of an attached first suture through the first one of the leaflets;
piercing a second one of the leaflets of the native heart valve;
moving a second anchor and a portion of an attached second suture through the second one of the leaflets;
removing the device from the heart;
pulling the first suture and the second suture proximally; and
locking the first suture and the second suture together.

2. The method of claim 1, wherein a paddle of the plurality of paddles includes an inner paddle portion and an outer paddle portion, and wherein the inner paddle portion and the outer paddle portion are deployed from the catheter delivery system in a fully open position where the outer paddle portion extends away from the inner paddle portion.

3. The method of claim 1, wherein the plurality of paddles are moved into the partially open position in a ventricle of the heart.

4. The method of claim 1, wherein a paddle of the plurality of paddles includes an inner paddle portion and wherein the moving of the device causes the inner paddle portion to engage a ventricular side of a leaflet of the native heart valve.

5. The method of claim 1, wherein the first anchor is collapsable and expandable.

6. The method of claim 5, wherein the first anchor is disposed in the catheter delivery system in a collapsed state.

7. The method of claim 6, further comprising moving the first anchor through the first one of the leaflets in the collapsed state.

8. The method of claim 7, further comprising expanding the first anchor on a ventricular side of the first one of the leaflets.

9. A method of treating a heart valve comprising:
inserting a first catheter into a heart;
deploying a device from the first catheter into a chamber of the heart in a fully open position, wherein:
the device has a plurality of paddles that each include an inner paddle portion and an outer paddle portion;
the outer paddle portion extends away from the inner paddle portion in the fully open position;
moving the plurality of paddles into a partially open position;
moving the device with the first catheter such that the inner paddle portions engage a ventricular side of leaflets of the heart valve;
piercing a first one of the leaflets of the heart valve with a second catheter;
moving a first anchor and a portion of an attached first suture through the second catheter and through the first one of the leaflets;
expanding the first anchor on a ventricular side of the first one of the leaflets;
piercing a second one of the leaflets of the heart valve;
moving a second anchor and a portion of an attached second suture through the second one of the leaflets;
expanding the second anchor on a ventricular side of the second one of the leaflets;
removing the device from the heart;
pulling the first suture and the second suture proximally through a suture locking device;
locking the first suture and the second suture together with the suture locking device.

10. The method of claim 9, wherein the first anchor is collapsable and expandable.

11. The method of claim 9, wherein the second catheter pierces the second one of the leaflets.

12. The method of claim 9, wherein the first anchor is disposed in the second catheter in a collapsed state.

13. The method of claim 9, wherein the first anchor is moved through the first one of the leaflets in a collapsed state.

14. The method of claim 9, wherein a lumen extends through the second catheter and the second catheter has a sharp tip.

15. The method of claim 9, wherein the first anchor and the second anchor are made from a shape memory alloy.

16. A system of treating a heart valve comprising:
a first catheter configured to be inserted into a chamber of a heart;
a removable device comprising a plurality of paddles, a paddle of the plurality of paddles comprising an inner paddle portion and an outer paddle portion;
wherein the plurality of paddles are configured to move between a fully open position, where the inner paddle portion and outer paddle portion extend away from each other, and a partially open position, where the inner paddle portion and the outer paddle portion are moved toward each other;
a second catheter disposed within the first catheter, the second catheter comprising a sharp distal end configured to pierce one or more of the leaflets of the native heart valve;
an implantable device comprising: a first anchor and an attached first suture, wherein:
the first anchor and the attached suture is adapted to move through the second catheter and through a first one of the leaflets of the native heart valve; and
the first anchor is adapted to be expanded on a ventricular side of the first one of the leaflets.

17. The system of claim 16, wherein the implantable device further comprises a second anchor and an attached second suture.

18. The system of claim 16, wherein the first catheter and the removable device are configured to be removed from the heart.

19. The system of claim 17, wherein the implantable device further comprises a suture locking device configured to lock the first suture and the second suture together.

20. The system of claim 16, wherein the first anchor is made from a shape memory alloy.
